(19) United States
(12) Patent Application Publication
(10) Pub. No.: US 2017/0303794 A1
(43) Pub. Date: Oct. 26, 2017

(54) OBJECT-INFORMATION ACQUISITION APPARATUS

(71) Applicant: CANON KABUSHIKI KAISHA, Tokyo (JP)

(72) Inventor: Nobuhito Suehira, Tokyo (JP)

(21) Appl. No.: 15/513,400

(22) PCT Filed: Sep. 24, 2015
(86) PCT No.: PCT/JP2015/004844

§ 371 Date: Mar. 22, 2017

(30) Foreign Application Priority Data
Sep. 29, 2014 (JP) 2014-199183

(51) Int. Cl. A61B 5/00; A61B 8/14; A61B 8/00; A61B 5/145

(52) U.S. Cl. CPC A61B 5/0095; A61B 8/4477; A61B 5/14542; A61B 5/7475; A61B 8/14

(57) ABSTRACT

It is sometimes difficult for an operator who is not well familiar with an apparatus to acquire a desired image.

An object-information acquisition apparatus includes a light source configured to emit light, a photoacoustic-wave detecting unit configured to detect photoacoustic waves generated when the light is applied an object, a measurement-mode selecting unit, and a measurement-condition determination unit configured to determine at least one of measurement conditions including a wavelength of the light to be emitted by the light source and a central reception frequency of the photoacoustic-wave detecting unit on the basis of the measurement mode selected by the measurement-mode selecting unit.

### TECHNICAL FIELD

The present invention relates to an object-information acquisition apparatus.

### BACKGROUND ART

One of optical imaging techniques, photoacoustic tomography (PAT), has recently been proposed. In PAT, pulsed light is delivered into an object, and photoacoustic waves generated from the biological tissue of the object that has absorbed the energy of the light transmitted and spread in the object are detected. Signals generated on the basis of the detected photoacoustic waves are processed to visualize information on the optical characteristic values of the internal part of the object.

PTL 1 discloses a PAT apparatus that allows selecting a process for acquiring an image generated on the basis of the detected photoacoustic waves.

### CITATION LIST

### Patent Literature

PTL 1: Japanese Patent Laid-Open No. 2014-140717

### SUMMARY OF INVENTION

### Technical Problem

However, PTL 1 does not describe a method for setting measurement parameters in detecting the photoacoustic waves.

With the PAT apparatus disclosed in PTL 1, when an operator who is not well familiar with the operation of the apparatus sets measurement parameters, such as the wavelength of pulsed light emitted to an object and the reception frequency of a transducer for detecting photoacoustic waves, it is sometimes difficult to acquire a desired image.

The present invention provides an object-information acquisition apparatus that allows even an operator who is not well familiar with the apparatus to easily acquire a desired image.

### Solution to Problem

An object-information acquisition apparatus according to a first aspect of the present invention includes a light source configured to emit light, a photoacoustic-wave detecting unit configured to detect photoacoustic waves generated by irradiation of an object with the light, a measurement-mode selecting unit configured to select a measurement mode, and a measurement-condition determination unit configured to determine at least one of measurement conditions including a wavelength of the light to be emitted by the light source and a central reception frequency of the photoacoustic-wave detecting unit on the basis of the measurement mode selected by the measurement-mode selecting unit.

An object-information acquisition apparatus according to a second aspect of the present invention includes a light source configured to emit light to an object, an acoustic-wave detecting unit configured to detect acoustic waves generated by irradiation of the object with the light, a storage unit configured to store a first parameter on a light source and a second parameter on an acoustic-wave detecting unit in accordance with a measurement mode, and a control unit configured to cause a selection window for an operator to select a measurement mode from a plurality of measurement modes stored in the storage unit. The control unit controls emission of the light from the light source and detection of the acoustic waves with the acoustic-wave detecting unit using the first and second parameters determined in accordance with a measurement mode selected by the operator.

Further features of the present invention will become apparent from the following description of exemplary embodiments with reference to the attached drawings.

### Advantageous Effects of Invention

According to some embodiments of the present invention, even an operator who is not well familiar with the apparatus can easily acquire a desired image.

### BRIEF DESCRIPTION OF DRAWINGS

FIG. 1 is a diagram showing the configuration of a photoacoustic tomography apparatus according to a first embodiment of the present invention.

FIG. 2 is a diagram showing a measurement-mode selection window according to the first embodiment of the present invention.

FIG. 3 is a flowchart of a measuring sequence according to the first embodiment of the present invention.

FIG. 4 is a diagram showing a measurement-mode selection window according to a second embodiment of the present invention.

### DESCRIPTION OF EMBODIMENTS

### First Embodiment

In this embodiment, a hand-held PAT apparatus in which an operator can manually operate a probe for detecting photoacoustic waves will be described by way of example.

### System Configuration

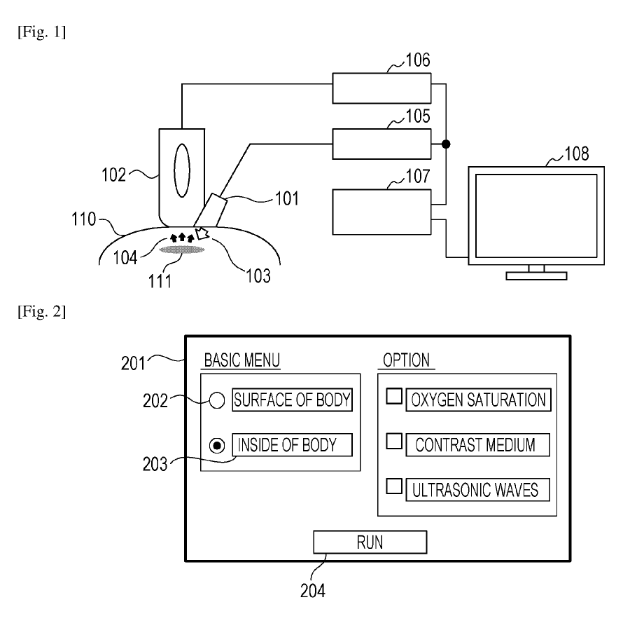

FIG. 1 shows the configuration of a PAT apparatus, which is an object-information acquisition apparatus according to this embodiment. This PAT apparatus includes a light-emitting unit 101 serving as a light source, an ultrasonic probe 102 serving as a photoacoustic-wave detecting unit, a light-emission control unit 105, a probe control unit 106, and an apparatus control unit 107.

### Light-Emitting Unit

The light-emitting unit 101 is a device that emits pulsed light to an object. The light-emitting unit 101 may be a laser light source capable of outputting high power, such as a solid-state laser, a gas laser, a dye laser, or a semiconductor laser. The light-emitting unit 101 is not limited to the laser light source but may be a light-emitting diode or a flash lamp. The emission timing, pulse width, intensity, and so on of the pulsed light are controlled by the light-emission control unit 105. The number of the light-emitting unit 101 does not need to be one; a plurality of light-emitting units may be used to irradiate the object from a plurality of directions to eliminate blind spots.

Applying light to the object for an adequately shorter time than thermal diffusion time and acoustic wave transmission time allows photoacoustic waves to be effectively generated. If the object is a living organism, the pulse width of the pulsed light generated from the light-emitting unit 101 may be about 10 to 50 nanoseconds. The wavelength of the pulsed light may be a wavelength at which the pulsed light is propagated to a region to be visualized in the object. Specifically, for a living organism, 700 nm or more and 1,100 nm or less are preferably. More specifically, the wavelength can be varied in the range of 720-880 nm using a titanium-sapphire laser, which is a solid-state laser. A dye laser with a wavelength of 580 nm is used as needed.

The light-emitting unit 101 may not include a light source; for example, light output from a light source provided outside the light-emitting unit 101 may be introduced.

### Ultrasonic Probe

When light 103 transmitted through a living organism is absorbed by an absorber 111, such as red blood cells, in the object, photoacoustic waves 104 are generated from the absorber 111. The generated photoacoustic waves 104 are received by the ultrasonic probe 102 including devices capable of detecting ultrasonic waves. The received signal is converted to an analog electrical signal. Thereafter, the analog electrical signal is transmitted to the probe control unit 106, where it is amplified by an amplifier of the probe control unit 106, and is then converted to a digital signal by an analog-to-digital converter. The obtained digital signal is transmitted to the apparatus control unit 107. The timing of reception of the ultrasonic waves is controlled by the apparatus control unit 107 so as to synchronize with the light emission of the light-emitting unit 101. The ultrasonic probe 102 serving as a photoacoustic-wave detecting unit is a single probe.

The ultrasonic probe 102 may be highly sensitive and have a wide frequency band. Examples of devices mounted to the ultrasonic probe 102 that meet the requirements include piezoelectric ceramics, such as lead zirconate titanate (PZT), a capacitive micromachined ultrasonic transducer (CMUT), and other transducers.

A photoacoustic wave receiving surface of the ultrasonic probe 102 may be either flat or curved along the external form of the object. In an example, 256 devices may be arrayed in a straight line at 2-mm pitch. The devices is not limited to be arrayed in a straight line but may be arrayed in two-dimensions or concentrically. Furthermore, the photoacoustic-wave receiving surface of the ultrasonic probe 102 may have a hemispherical form around which a plurality of devices are arrayed in a concentric form or a spiral form. The hemispherical receiving surface is suitable when the object is a breast of a living organism. The photoacoustic-wave receiving surface may have a cylindrical form or a semicylindrical form on which a plurality of devices are arrayed. The cylindrical or semicylindrical receiving surface is suitable when the object is an arm or a leg of a living organism.

In this embodiment, the central reception frequency of the ultrasonic probe 102 can be varied, for example, within 2-20 MHz. The central reception frequency of the photoacoustic waves detected by the ultrasonic probe 102 can be changed by changing the central reception frequency. In this specification, the central reception frequency of the ultrasonic probe 102 is a frequency at which the ultrasonic probe 102 has high sensitivity, typically, the highest sensitivity. A plurality of devices having different central reception frequencies may be disposed on the receiving surface of the ultrasonic probe 102, so that the central reception frequency of the ultrasonic probe 102 can be changed by switching between devices that use an obtained electrical signal. The central reception frequency may be switched on the receiving surface of the ultrasonic probe 102 on which devices with low reception frequencies and devices with high reception frequencies are disposed. The electrical signal obtained from the photoacoustic waves is sampled at a sampling frequency of 50 MHz, and 1,024 samples are obtained. The digital signal obtained using the analog-to-digital converter has signed 12 bits.

### Apparatus Control Unit

The apparatus control unit 107 serves as a measurement-mode selecting unit that selects a measurement mode: controls the light-emitting unit 101 and the ultrasonic probe 102; and reconstructs an image based on the photoacoustic waves detected by the ultrasonic probe 102, that is, generates image data. In this embodiment, the apparatus control unit 107 also serves as a measurement-condition determination unit and an image processing unit. Furthermore, the apparatus control unit also serves as a display control unit that causes a display device 108 to display an image based on the generated image data. The apparatus control unit 107 includes a user interface, allowing change of measurement parameters, start and termination of the measurement, selection of a method of image processing, storage of object information and an image, analysis of data, and so on to be selected in accordance with an instruction from the operator. The apparatus control unit 107 may include a display as a user interface, so that the operator can perform the above selection with an operation menu on the display. The display of the apparatus control unit 107 may be a touch panel.

An image is displayed on the display device 108 on the basis of the image data generated by the apparatus control unit 107. The unit for reconstructing an image may be either a computer independent of the apparatus control unit 107 and including a CPU, a main storage unit, and an auxiliary storage unit, or specifically designed hardware.

The apparatus control unit 107 may include a display control unit that controls display on a display device, a light-source control unit that controls the light source, and a detecting-unit control unit that controls the acoustic-wave detecting unit.

### Selection Window 1

FIG. 2 shows a measurement-mode selection window 201 according to this embodiment. The selection window 201 may be displayed on the display device 108 or may be displayed on the display of the apparatus control unit 107 if provided. In this embodiment, two choices “Basic Menu” and “Option” are displayed on a measurement menu from which the operator can select. The “Basic Menu” field allows the operator to select “Surface of Body” or “Inside of Body”. The “Option” field allows the operator to select at least one of “Oxygen Saturation”, “Contrast Medium”, and “Ultrasonic Waves”.

The reason why one of “Surface of Body” and “Inside of Body” can be selected in “Basic Menu” is that the wavelength of pulsed light applied to the object and the reception frequency of the photoacoustic waves can be roughly classified depending on whether the measurement is for the surface of body or the inside of body. In this embodiment, the operator selects measurement of the surface of body or measurement of the inside of body on the “Basic Menu” using a radio button 202. The operator further selects options, such as “Oxygen Saturation”, “Contrast Medium”, and “Ultrasonic Waves” as necessary. The “Oxygen Saturation” is an option item for calculating and displaying oxygen saturation in a image reconstruction region of the object. The “Contrast Medium” is an option item that is selected when an object given a contrast medium is to be measured. The “Ultrasonic Waves” is an option item for acquiring an ultrasonic echo image by applying ultrasonic waves to an object and detecting reflected ultrasonic waves. The source of the ultrasonic waves applied to the object when “Ultrasonic Waves” is selected may be disposed on the ultrasonic probe 102. When a “Run” button 204 is pressed after “Basic Menu” and “Option” are selected, a source wavelength and a central reception frequency of photoacoustic waves suitable for the measurement mode are automatically determined by the apparatus control unit 107. Among the choices displayed on the selection window 201, “Basic Menu” is an exclusive or, while “Option” allows the operator to select a plurality of choices at the same time.

Suppose measurement of the oxygen saturation of a breast of a living organism using the PAT apparatus according to this embodiment. The operator selects “Inside of Body” and “Oxygen Saturation” on the selection window 201 and presses the “Run” button 204. To measure the oxygen saturation, pulsed lights with two different wavelengths need to be applied to the object. In this embodiment, the apparatus control unit 107 automatically sets the source wavelengths of the pulsed lights to 756 nm and 797 nm, and the central reception frequency of the ultrasonic probe 102 to 3 MHz. Since a breast needs to be measured to a depth of about 4 cm, near-infrared light having a long light penetration depth is used for source pulsed light. The central reception frequency of the ultrasonic probe 102 is set to a low frequency of about 3 MHz to draw a relatively large structure, such as a tumor or a thick blood vessel in a breast. In an example of a method for displaying an image acquired by this measurement, oxygen saturation is superimposed on a sound level distribution for display. Another method of display is displaying an image showing a sound level distribution and an image showing oxygen saturation side by side. A method of display may either be automatically determined by the apparatus control unit 107 in response to selection of a measurement mode or be selected by the operator.

Suppose measurement on a skin of a living organism using the PAT apparatus according to this embodiment. In a case where the measurement is executed with only “Surface of Body” selected and all the items of “Option” unselected by the operator on the selection window 201, the apparatus control unit 107 automatically sets the source wavelength of the pulsed light to 580 nm, and the central reception frequency of the ultrasonic probe 102 to 20 MHz. This is because observation in the range of around 5 mm is enough to perform measurement on a skin or the like. This allows visible light with a wavelength shorter than that selected for measurement on “Inside of Body”, that is, visible light with a short penetration length, to be used for the source wavelength. In contrast, the photoacoustic waves needs to be set to a high central frequency because photoacoustic waves have to be detected at high resolution. Since the central reception frequency differs between measurement on “Surface of Body” and measurement on “Inside of Body”, the resolution of measurement differs. This results in different pixel resolutions of the images. Therefore, for measurement on “Surface of Body”, interpolation may be performed to prevent the difference in resolution from being viewed in the displayed images. In this specification, “Surface of Body” is a relatively shallow region from the surface of the object to a depth of about 1 cm.

When “Contrast Medium” in “Option” is selected, and the contrast medium is indocyanine green (ICG), the source wavelength is 780 nm. When “Contrast Medium” is selected on the selection window 201, a menu that prompts the operator to select the kind of contrast medium may be further displayed. Thus, the apparatus control unit 107 selects a source wavelength suitable for the contrast medium given to the object. To measure a change with time, time-series images are displayed as a method of displaying images. An image displayed on the display device 108 may be switched in sequence in a slide show format, or alternatively, a plurality of images may be displayed side by side in one window. A difference from the first image may be displayed to make it easy to know a change with time.

When “Ultrasonic Waves” and “Oxygen Saturation” in “Option” are selected, an image showing oxygen saturation may be interposed on an image acquired by measuring ultrasonic waves, of ultrasonic waves applied by the ultrasonic probe 102, reflected by the object. In this case, the ultrasonic probe 102 switches between reception of photoacoustic waves and transmission and reception of ultrasonic waves for usage.

For more detailed setting, an item button 203 can be used. For example, when a “Surface of Body” button is pressed, setting for a part, such as a face, an arm, or a neck, and an observation target, such as a melanoma or a tumor, can be performed. Although the setting in this case is classified into “Surface of Body” and “Inside of Body”, a selection window for measurement parts, such as a face, an arm, a neck, and a breast, may be displayed. For each of the parts, “Option” as shown in FIG. 2 may be set.

A plurality of ultrasonic probes 102 with different central reception frequencies may be provided so that an ultrasonic probe 102 to be used can be changed depending on the details of “Basic Menu” and “Option” selected by the operator. The ultrasonic probe 102 may be detachable so that an ultrasonic probe 102 to be used can be replaced depending on the details of “Basic Menu” and “Option” selected by the operator. In such cases, a message that tells an ultrasonic probe 102 to be used to the operator may be displayed on at least one of the display of the apparatus control unit 107 and the display device 108. In the case where the PAT apparatus includes the plurality of ultrasonic probes 102 or is configured to replace the ultrasonic probe 102, the individual ultrasonic probes 102 may be differently shaped in the form of the receiving surface to the measurement target to make a good contact therewith.

The above example has been described on the assumption that the object is a living organism. In using the PAT apparatus for measurement of an object other than a living organism, a window for selecting the kind of the object may be displayed before the selection window 201 is displayed. When a living organism is selected as the object, the selection window 201 shown in FIG. 2 is displayed.

### Measuring Process 1

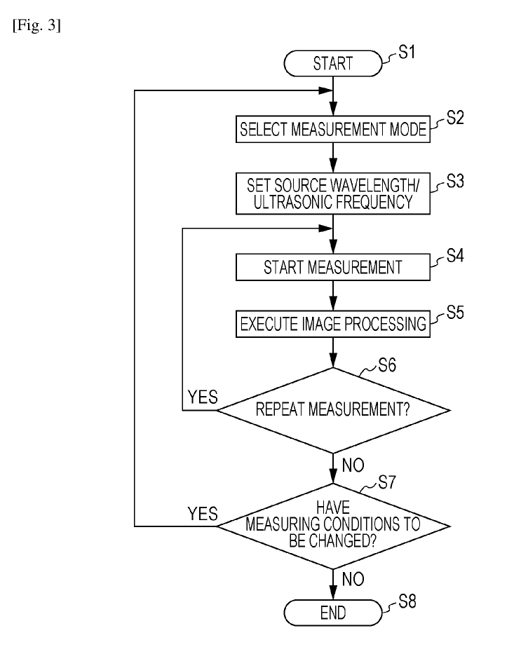

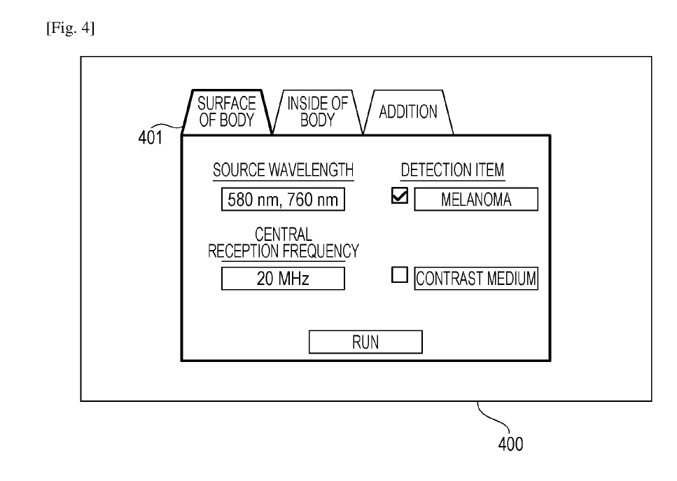

FIG. 3 is a flowchart of a measuring sequence of this embodiment. Suppose a case where both of oxygen saturation and an ultrasonic echo image are acquired for a breast to be observed. The object measuring sequence is started from Step S1. The apparatus control unit 107 causes the selection window 201 to be displayed on its display or the display device 108.

Step S2 is the process of selecting a measurement mode. At step S2, the apparatus control unit 107 displays the selection window 201 on the display of the apparatus control unit 107 or the display device 108 to prompt the operator to select a measurement mode. In response, the operator selects a measurement mode. In this embodiment, the operator selects “Inside of Body” in “Basic Menu” and selects “Oxygen Saturation” and “Ultrasonic Waves” in “Option”.

Step S3 is the process of determining measurement conditions corresponding to the measurement mode selected at step S2. Since “Inside of Body”, “Oxygen Saturation”, and “Ultrasonic Waves” are selected at step S2, the apparatus control unit 107 sets two source wavelengths 756 nm and 797 nm for the pulsed light and sets the central reception frequency of the photoacoustic waves detected by the ultrasonic probe 102 to 3 MHz. When a plurality of light sources are used, the optical path of light emitted from a light source corresponding to a wavelength selected as necessary is switched so that the light is applied to a target region of the object. At this step, the ultrasonic wave circuit is switched between transmission and reception of ultrasonic waves. After completion of the setting, the process goes to step S4.

The measurement is started from step S4. Before the measurement, the operator applies gel for acoustic coupling to the target region and brings the ultrasonic probe 102 of the PAT apparatus into contact therewith. The measurement is started with the ultrasonic probe 102 in contact. First, ultrasonic echo measurement is performed using the ultrasonic probe 102 to search for a desired measurement region from which photoacoustic waves are to be obtained. The ultrasonic echo measurement is performed on the desired region, and then photoacoustic measurement is performed. The switching from the ultrasonic echo measurement to the photoacoustic measurement may be performed in accordance with an instruction from the operator using an operation button. The ultrasonic echo measurement and the photoacoustic measurement may be automatically switched therebetween. For the ultrasonic echo measurement, the probe control unit 106 transmits and receives ultrasonic waves and sets the central reception frequency to 12 MHz, and for photoacoustic waves, the probe control unit 106 sets the central reception frequency to 3 MHz. In other words, the ultrasonic probe 102 can operate also as an ultrasonic-wave generating unit. The photoacoustic measurement is performed for both of a wavelength of 756 nm and a wavelength of 797 nm of the pulsed light. In this case, the ultrasonic waves probe 102 switches the central reception frequency between 3 MHz and 12 MHz using an electrical filter.

At step S5, an image is displayed. Data obtained until step S4 is an ultrasonic echo image and photoacoustic images acquired from pulsed light with a frequency of 756 nm and pulsed light with a frequency of 797 nm. An image of oxygen saturation can be calculated from image data on which the photoacoustic images of 756 nm and 797 nm are based. As a method of displaying an image, the image of the oxygen saturation is superimposed on the ultrasonic echo image and is displayed on the display device 108.

At step S6, the operator determines whether a repetition of the measurement is required. If the operator determines that a desired image is acquired by checking the image, the operator inputs an instruction to terminate the measurement to complete the measurement. The completion of the measurement can be input via, for example, the user interface of the apparatus control unit 107. If the target object is a breast, the same measurement is performed on the other breast of the identical object as needed. If the operator determines that repeated measurement is necessary, or the same measurement is to be performed on the other breast, the process returns to step S4 for measurement.

At step S7, the operator determines whether the measurement conditions need to be changed. If measurement on, not the internal part of the breast, but another part, such as a skin, is needed, the process returns to step S2 for selecting a measurement mode. Also for a case where measurement is performed on the same part under conditions suitable for, for example, an object given a contrast medium, the process returns to step S2 for selecting a measurement mode. If there is no need to change the measurement conditions, the process goes to step S8.

At step S8, the measuring sequence is completed.

In this embodiment, since measurement conditions can be automatically determined when a measurement mode is selected, as described above, a desired image can easily be acquired.

### Second Embodiment

A second embodiment is an object-information acquisition apparatus in which measurement parameters can be set using a tab-format selection window. Differences from the first embodiment will be mainly described hereinbelow.

### Selection Window 2

FIG. 4 shows a measurement-mode selection window 400 according to this embodiment. Tabs 401 are used to select the details of setting. In this case, an “Addition” tab is provided in addition to a “Surface of Body” tab and an “Inside of Body” tab for a measurement mode, allowing the operator to set desired measurement parameters.

The operator first selects one of the “Surface of Body” tab, the “Inside of Body” tab, and the “Addition” tab in the selection window 400. When the operator selects the “Surface of Body” tab, “580 nm” is displayed in a “Source Wavelength” field as the source wavelength of the pulsed light, and “20 MHz” is displayed in a “Central Reception Frequency” field as the central reception frequency of the ultrasonic probe 102. These two values are preset values that are automatically determined by selecting the “Surface of Body” tab. When the operator further selects “Melanoma” as a measurement mode from “Detection Item”, “760 nm” is further displayed in the “Source Wavelength” field. Since FIG. 4 shows a selection window when “Melanoma” is selected, “580 nm, 760 nm” are displayed in the “Source Wavelength” field. When the operator selects “Contrast Medium”, a different wavelength is displayed in the “Source Wavelength” field. For example, when indocyanine green, described above, is used as a contrast medium, “780 nm” is further displayed in the “Source Wavelength” field.

Also when the operator selects the “Inside of Body” tab, preset values are displayed in the “Source Wavelength” field and the “Central Reception Frequency” field as for the “Surface of Body”, and when the operator further selects “Detection Item”, a source wavelength is added in the “Source Wavelength” field.

When the operator selects the “Addition” tab, another measurement, for example, execution of ultrasonic echo measurement can be selected in addition to measurement of photoacoustic waves for “Surface of Body” or “Inside of Body”.

### Measuring Process 2

An example of a measuring sequence for measurement on melanoma will be described with reference to the flowchart in FIG. 3.

The object measuring sequence is started from step S1. The apparatus control unit 107 causes the selection window 400 to be displayed on its display or the display device 108.

At step S2, a measurement mode is selected on the selection window 400 in FIG. 4. In this embodiment, the operator selects the “Surface of Body” tab and selects “Melanoma” in “Detection Item” as an option.

Step S3 is the process of determining measurement conditions corresponding to the measurement mode selected at step S2. Since “Surface of Body” and “Melanoma” are selected at step S2, the apparatus control unit 107 sets two source wavelengths, 580 nm and 760 nm, for the pulsed light and sets the central reception frequency of the photoacoustic waves detected by the ultrasonic probe 102 to 20 MHz.

The measurement is started from step S4. Before the measurement, the operator applies gel for acoustic coupling to the target region and brings the ultrasonic probe 102 into contact with a desired position. The photoacoustic measurement is performed for each of the source wavelengths 580 nm and 760 nm of the pulsed light. The switching between the source wavelengths is achieved by switching between the optical paths of a dye laser and a titanium-sapphire laser, for example.

At step S5, an image is displayed. A method for displaying an image is selected which allows the relationship between melanoma and its peripheral blood vessels to be viewed from measurement images acquired at the source wavelengths of 580 nm and 760 nm of the pulsed light, and the images are displayed in a superimposed manner

At step S6, the operator determines whether a repetition of the measurement is required. If the operator determines that a desired image is acquired by checking the image, the measurement is completed. If the operator determines that repeated measurement is necessary, or the same measurement is to be performed on another part, the process returns to step S4 for measurement.

At step S7, the operator determines whether the measurement conditions need to be changed. If measurement on another par is needed, the process returns to step S2 for selecting a measurement mode. If there is no need to perform measurement on another part, the process goes to step S8.

At step S8, the measuring sequence is completed.

Also in this embodiment, since measurement conditions can be automatically determined when a measurement mode is selected, as described above, a desired image can easily be acquired.

In the above embodiments, the hand-held PAT apparatus in which an operator can move with the ultrasonic probe 102 in hand has been described as an example. However, the configuration of the PAT apparatus is given for mere illustration and is not intended to limit the invention. A floor-mounted object-information acquisition apparatus or an ultrasonic probe that can be moved on a predetermined path or within a predetermined range may be employed.

In the above embodiments, the central reception frequency of the ultrasonic probe 102 is switched depending on a selected measurement mode. A conceivable method for achieving the switching is providing a plurality of probes with different central reception frequencies and using a probe corresponding to a selected measurement mode. In the case where a plurality of probes are provided, the apparatus control unit 107 may activate only a probe to be used in a selected measurement mode. In this case, the activated probe may be displayed on the display device 108 to increase the convenience of the operator.

The ultrasonic probe 102 may be interchangeable so that an ultrasonic probe 102 having a central reception frequency for a selected measurement mode can be used.

In the above embodiments, the apparatus control unit 107 may be configured to determine the pulse width of the light emitted by the light-emitting unit 101 depending on a selected measurement mode.

It will be appreciated that the above embodiments are given for mere illustration, and the elements of the embodiments can be combined without departing from the spirit of the present invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-199183, filed Sep. 29, 2014, which is hereby incorporated by reference herein in its entirety.

### INDUSTRIAL APPLICABILITY

The above object-information acquisition apparatus can be used as a medical diagnostic imaging apparatus when the object is a biological substance. Specifically, the apparatus can image the distribution of optical characteristic values in a living organism and the density distribution of substances constituting the biological tissue acquired from the information to make a diagnosis of a tumor or a vascular disease or monitor a chemical treatment over time.

The apparatus can also be applied o a nondestructive examination on a nonliving substance.

The above embodiments show a case where the wavelength of pulsed light output from the light-emitting unit 101 serving as a light source and the central reception frequency of acoustic waves detected by the ultrasonic probe 102 serving as an acoustic-wave detecting unit are determined in advance.

Instead of or in addition to the wavelength output from the light source, another condition on the light source may be determined in advance. Instead of or in addition to the central reception frequency detected by the ultrasonic probe 102, another condition on the ultrasonic probe 102 may be determined in advance. Specifically, a storage unit that stores first parameters on the light source (for example, a wavelength, a pulse width, an amplitude, and a pulse interval) and second parameters on the ultrasonic probe 102 (for example, a central reception frequency, a sampling frequency, and a sampling interval) may be provided. For example, the storage unit may store a table in which the first and second parameters are associated with measurement modes. If conditions on the light source ad the acoustic-wave detecting unit are determined in advance for each measurement mode, various conditions can be automatically set when the operator selects a measurement mode.

The present invention can also be implemented by supplying a program for implementing one or more functions of the above embodiments to a system or apparatus via a network or a storage medium and by reading and executing the program with one or more processors of a computer of the system or apparatus. The present invention can also be implemented by a circuit that implements one or more functions, for example, an application specific integrated circuit (ASIC).

### REFERENCE SIGNS LIST

101 Light-emitting unit

102 Ultrasonic probe

105 Light-emission control unit

106 Probe control unit

107 Apparatus control unit

108 Display device

110 Object

1. An object-information acquisition apparatus comprising:
a light source configured to emit light;
a photoacoustic-wave detecting unit configured to detect photoacoustic waves generated by irradiation of an object with the light;
a measurement-mode selecting unit configured to select a measurement mode; and
a measurement-condition determination unit configured to determine at least one of measurement conditions including a wavelength of the light to be emitted by the light source and a central reception frequency of the photoacoustic-wave detecting unit on the basis of the measurement mode selected by the measurement-mode selecting unit.

2. The object-information acquisition apparatus according to claim 1, wherein as the measurement mode, a part of the object can be selected.

3. The object-information acquisition apparatus according to claim 2, wherein the part of the object includes a body surface or an inside of the object.

4. The object-information acquisition apparatus according to claim 2, wherein the part of the object includes at least one of a face, a neck, an abdomen, a breast, and an arm.

5. The object-information acquisition apparatus according to claim 2, wherein in the measurement mode, at least one of oxygen saturation of the object, an ultrasonic echo image of the object, and an image of the object given a contrast medium can be further selected.

6. The object-information acquisition apparatus according to claim 1, wherein the measurement-condition determination unit further determines a pulse width of the light to be emitted by the light source in accordance with the selected measurement mode.

7. The object-information acquisition apparatus according to claim 1, wherein the photoacoustic-wave detecting unit includes a plurality of probes having different central reception frequencies from one another.

8. The object-information acquisition apparatus according to claim 7, wherein the measurement-condition determination unit selects a probe to be activated from the plurality of probes in accordance with the selected measurement mode.

9. The object-information acquisition apparatus according to claim 1, wherein the measurement-mode selecting unit selects the measurement mode in accordance with an operator's operation.

10. The object-information acquisition apparatus according to claim 8, wherein the probe to be activated is displayed on a display device.

11. The object-information acquisition apparatus according to claim 1, wherein the photoacoustic-wave detecting unit includes an interchangeable probe.

12. The object-information acquisition apparatus according to claim 1, further comprising:
an ultrasonic-wave generating unit configured to generate ultrasonic waves,
wherein in the measurement mode, detection of ultrasonic waves reflected from the object with the photoacoustic-wave detecting unit can be selected.

13. The object-information acquisition apparatus according to claim 1,
wherein the light source includes a plurality of light-emitting devices that emits light having different wavelengths from one another; and
wherein the measurement-condition determination unit determines a light-emitting device that emits light to the object from the plurality of light-emitting devices in accordance with the selected measurement mode.

14. The object-information acquisition apparatus according to claim 1, wherein
the measurement-mode selecting unit includes a display control unit that causes a selection window that prompts an operator to select the measurement mode to be displayed on a display device,
wherein when the measurement mode is selected, the display control unit causes the display device to display the wavelength of the light to be emitted by the light source and the central reception frequency of the photoacoustic-wave detecting unit according to the selected measurement mode.

15. The object-information acquisition apparatus according to claim 14, further comprising:
an image processing unit configured to generate image data based on the detected photoacoustic waves,
wherein the display control unit determines a method for displaying an image based on the image data on the display device in accordance with the selected measurement mode.

16. The object-information acquisition apparatus according to claim 14, further comprising the display device.

17. An object-information acquisition apparatus comprising:
a light source configured to emit light to an object;
an acoustic-wave detecting unit configured to detect acoustic waves generated by irradiation of the object with the light;
a storage unit configured to store a first parameter on a light source and a second parameter on an acoustic-wave detecting unit in accordance with a measurement mode; and
a control unit configured to cause a selection window for an operator to select a measurement mode from a plurality of measurement modes stored in the storage unit,
wherein the control unit controls emission of the light from the light source and detection of the acoustic waves with the acoustic-wave detecting unit using the first and second parameters determined in accordance with a measurement mode selected by the operator.
